SEQUENCE LISTING (12 sequences)

SEQ ID NO: 1 - DNA, 48 bp, Unknown
  Bxb1 mycobacteriophage
ggtttgtctggtcaaccaccgcggtctcagtggtgtacggtacaaacc

SEQ ID NO: 2 - DNA, 48 bp, Artificial Sequence
  Synthetic
ggtttgtctggtcaaccaccgcggactcagtggtgtacggtacaaacc

SEQ ID NO: 3 - DNA, 38 bp, Unknown
  Bxb1 mycobacteriophage
ggcttgtcgacgacggcggtctccgtcgtcaggatcat

SEQ ID NO: 4 - DNA, 38 bp, Artificial Sequence
  Synthetic
ggcttgtcgacgacggcggactccgtcgtcaggatcat

SEQ ID NO: 5 - DNA, 150 bp, Artificial Sequence
  Synthetic
  misc_feature (1)..(3): phosphorothioate bond
  misc_feature (148)..(150): phosphorothioate bond
gattgaggaaggaactggtgtagcctagctggtctgagcatctctgccatgcggtttgtctggtcaaccaccgcggactcagtggtgtacggtacaaaccggctccctgtgtgtctgatcttgctaggaccgcttataggtaaggagaaa

SEQ ID NO: 6 - DNA, 21 bp, Artificial Sequence
  Synthetic
gacacacagggagccgcatgg

SEQ ID NO: 7 - RNA, 18 nt, Artificial Sequence
  Synthetic
cgaggcauggucccagcc

SEQ ID NO: 8 - RNA, 18 nt, Artificial Sequence
  Synthetic
uugcagcccgguugcugc

SEQ ID NO: 9 - DNA, 289 bp, Artificial Sequence
  Synthetic
tgcccaacccaagttcaaaggctgatgagagagaaaaactcatgaagagattttactctagggaaagttgctcagtggatgggatcttggcgcacggggaaagatgtccagctcctcctggctccttctcagccttgttgctgttactactgctcagtccctcaccgaggaaaatgccaagacatttttaaacaactttaatcaggaagctgaagacctgtcttatcaaagttcacttgcttcttggaattataatactaacattactgaagaaaatgcccaaaagatg

SEQ ID NO: 10 - DNA, 2442 bp, Artificial Sequence
  Synthetic
atggcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctcctttgattacaaggatgacgacgataagtga

SEQ ID NO: 11 - protein, 813 aa, Artificial Sequence
  Synthetic
MASSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSFDYKDDDDK

SEQ ID NO: 12 - DNA, 84 bp, Artificial Sequence
  Synthetic
gcagccactatctgtagcctcattattgcatgccaactgggtgcatgggccagggcccctggcttgcatcgaggcctgccacag

(19) United States
(12) Patent Application Publication
(10) Pub. No.: US 2023/0287459 A1
(43) Pub. Date: Sep. 14, 2023

(54) SINGLE GENERATION TARGETED GENE INTEGRATION

(71) Applicant: The Jackson Laboratory, Bar Harbor, ME (US)

(72) Inventors: Michael V. Wiles, Bar Harbor, ME (US); Benjamin E. Low, Bar Harbor, ME (US); Vishnu Hosur, Bar Harbor, ME (US)

(73) Assignee: The Jackson Laboratory, Bar Harbor, ME (US)

(21) Appl. No.: 18/019,999

(22) PCT Filed: Aug. 6, 2021
(86) PCT No.: PCT/US2021/044979

§ 371 Date: Feb. 6, 2023

Related U.S. Application Data
(60) Provisional application No. 63/063,147, filed on Aug. 7, 2020.

(51) Int. Cl. C12N 15/90; C12N 9/48; C12N 15/11; C12N 9/22; C12N 15/85; C12N 5/073

(52) U.S. Cl. CPC C12N 15/907; C12N 9/485; C12Y 304/17023; C12N 15/11; C12N 9/22; C12N 15/8509; C12N 5/0604; C12N 2310/20; C12N 2800/80; C12N 2800/30; C12N 2510/00

(57) ABSTRACT

The present disclosure provides methods and compositions for high frequency, targeted mammalian transgenesis using, for example, a two-step, two-stage process that enables integrating anywhere in the mammalian genome large pieces of nucleic acid in single generation.

### RELATED APPLICATION

This application claims the benefit under 35 U.S.C. § 119(e) of U.S. provisional application No. 63/063,147 filed Aug. 7, 2020 which is incorporated by reference herein in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Aug. 5, 2021, is named J022770088WO00-SEQ-NTJ, and is 13,140 bytes in size.

### GOVERNMENT LICENSE RIGHTS

The invention was made with government support under R21 OD027052 awarded by National Institutes of Health. The government has certain rights in the invention.

### BACKGROUND

The genome engineering revolution continues to drive rapid modification by the addition of DNA to the genomes of animals, for example creating more elegantly complex strains of mutant mice faster than ever before. While small genome modifications are becoming simpler and more efficient to mediate via programmable nuclease-based systems, the use of homologous recombination for precise insertion of large donor DNA remains challenging. For example, generating humanized mice requires incorporating control regions of a gene in order to recapitulate the intended expression pattern and function. The current approach using random and inherently shambolic transgenesis suffers from low efficiency, partial/incomplete integration, and multicopy concatemerization. Furthermore, such insertions often deposit into active loci, resulting in deleterious positional effects on transgene expression and the unintentionally disrupted endogenous gene(s). Consequently, large construct transgenic projects often require substantial characterization time and extra rounds of breeding, increasing the cost and time it takes to generate animal models of disease.

### SUMMARY

The present disclosure provides, in some aspects, an efficient, targeted transgenesis technology that enables integrating anywhere in the mammalian genome large pieces (e.g., greater than 3 kb) of nucleic acid. Advantageously, this technology may be used, for example, to rapidly develop model systems, in any genetic background, for gene-specific temporal (e.g., developmental) and spatial (e.g., cell-specific) control of transgene expression.

Some aspects of the present disclosure provide methods comprising: (a) delivering to a zygote a nucleic acid comprising a first integrase attachment site; (b) culturing the zygote to produce a multi-cell embryo comprising the first integrase attachment site in the genome of the embryo; and (c) delivering to the embryo a nucleic acid comprising a second integrase attachment site and a sequence encoding a product of interest to produce an engineered embryo comprising the second integrase attachment site.

In some embodiments, the method further comprises delivering to the embryo a cognate integrase or a nucleic acid encoding a cognate integrase. In other embodiments, a nucleic acid encoding a cognate integrase is integrated into the genome of the zygote (e.g., the zygote is produced from one or more mouse line(s) engineered to encode the cognate integrase in its genome).

In some embodiments, the methods further comprise implanting the engineered embryo (e.g., two-cell stage embryo) into a pseudopregnant female mammal capable of giving birth to a progeny mammal.

In some embodiments, the delivering to the zygote is via electroporation. In other embodiments, the delivering to the zygote is via microinjection. In some embodiments, the delivering to the embryo is via microinjection. In other embodiments, the delivering to the embryo is via electroporation.

In some embodiments, the multi-cell embryo is a two-cell embryo. In some embodiments, each cell of the two-cell embryo is microinjected.

In some embodiments, the integrase is Bxb1. Other integrases may be used.

In some embodiments, the first integrase attachment site of (a) is a Bxb1 attP attachment (or modified version), and the second integrase attachment site of (c) is a Bxb1 attB attachment site (or modified version). In other embodiments, the first integrase attachment site of (a) is a Bxb1 attB attachment site, and the second integrase attachment site of (c) is a Bxb1 attP attachment site. It should be understood that the term “attP attachment site” includes the sequence of SEQ ID NO: 1 and modified versions of that sequence, such as the sequence of SEQ ID NO: 2. Likewise, the term “attB attachment site” includes the sequence of SEQ ID NO: 3 and modified versions of that sequence, such as the sequence of SEQ ID NO: 4.

In some embodiments, the first integrase attachment site of (b) is operably linked to an endogenous promoter of a gene of interest. In some embodiments, the first integrase attachment site of (b) is upstream from (5′) and in frame with a transcriptional start codon. In other embodiments, the first integrase attachment site of (b) is downstream from (3′) and in frame with a transcriptional start codon.

In some embodiments, the first integrase attachment site of (a) is flanked by nucleotide sequences homologous to nucleotide sequences in the genome of the zygote.

In some embodiments, the nucleic acid of (i) is a vector backbone-free DNA minicircle.

In some embodiments, the nucleic acid of (ii) is a messenger RNA (mRNA).

In some embodiments, the method further comprises delivering to the zygote a programmable nuclease.

In some embodiments, the programmable nuclease is an RNA-guided nuclease and the method further comprises delivering to the zygote (i) an RNA-guided nuclease or a nucleic acid encoding the RNA-guided nuclease and (ii) a guide RNA (gRNA) targeting the gene of interest.

In some embodiments, the RNA-guided nuclease and the gRNA form a ribonucleoprotein.

In some embodiments, the RNA-guided nuclease is Cas9. In other embodiments, the programmable nuclease is a zinc finger nuclease (ZFN). In yet other embodiments, the programmable nuclease is a transcription activator-like effector nuclease (TALEN). Other gene editing systems may be used.

In some embodiments, the zygote is a mammalian zygote. In some embodiments, the zygote is a mammalian zygote is a non-human zygote (e.g., a commercial food animal, such as a cow, a pig, a sheep, a goat, or a chicken). In some embodiments, the mammalian zygote is a rodent zygote. The rodent zygote may be, for example, a rat zygote or a mouse zygote. In some embodiments, the mouse zygote is a NOD.Cg-Prkdc^{scid} I12rg^{tmIWjl}/SzJ (NSG®) mouse zygote.

In some embodiments, the methods further comprise breeding progeny mammals birthed by the pseudopregnant female mammal.

In some embodiments, the rodent zygote is a mouse zygote and the endogenous promoter is a mouse albumin promoter. In some embodiments, the product of interest is human albumin.

In some embodiments, the rodent zygote is a mouse zygote and the endogenous promoter is a mouse host cell receptor angiotensin-converting enzyme 2 (mAce2) promoter. In some embodiments, the product of interest is human host cell receptor angiotensin-converting enzyme 2 (huACE2).

SARS-CoV-2 enters the human body through ACE2 receptors. The S glycoprotein attaches to the ACE2 receptor on host cells, resulting in fusion of SARS-CoV-2 with the host cell. Following fusion, the type II transmembrane serine protease (TMPRSS2) present on the surface of the host cell clears the ACE2 receptor and activates the receptor-attached S glycoproteins, leading to a conformational change that allows the virus to enter the host cell (Rabi et al. Pathogens 2020; 9: 231). Thus, ACE2 and TMPRSS2 are the main determinants of viral entry.

In rodents, SARS-CoV-2 does not bind efficiently to endogenous ACE2 protein. Thus, to provide model systems that recapitulates SARS-CoV-2 infection in humans, the present disclosure provides, in some aspects, transgenic rodent models (such as mouse models) engineered to express human ACE2 protein (huACE2).

Other aspects of the present disclosure provide progeny mammals produced by any one of the foregoing methods, wherein the progeny mammal is a rodent. In some embodiments, the rodent is a mouse.

Yet other aspects of the present disclosure provide rodents comprising the engineered embryo produced by a method of any one of the preceding paragraphs.

Still other aspects of the present disclosure provide a method comprising administering a candidate prophylactic or therapeutic agent to the progeny mammal described above.

In some embodiments, the candidate agent is convalescent human serum, a human vaccine, or an antimicrobial agent, optionally an antibacterial agent and/or an antiviral agent. In some embodiments, the method further comprises infecting the mouse with SARS-CoV-2. In some embodiments, the method further comprises assessing efficacy of the agent for preventing SARS-CoV-2 infection and/or development of COVID-19.

In some embodiments, the product of interest is a programmable nuclease. In some embodiments, the product of interest is an RNA-guided nuclease. In some embodiments, the product of interest is Cas9. In some embodiments, the method further comprises delivering to the embryo a guide RNA (gRNA) targeting a nucleotide sequence of interest. In some embodiments, the nucleotide sequence of interest is an interferon regulatory factor 5 (Irf5) gene. In some embodiments, the product of interest is a zinc finger nuclease (ZFN). In some embodiments, the product of interest is a transcription activator-like effector nuclease (TALEN).

In some embodiments, the first integrase attachment site is located in a safe harbor locus of the genome. In some embodiments, the safe harbor locus is a ROSA26 locus, an AAVS1 locus, a Hip11 locus, an Hprt locus, or a Tigre locus.

### BRIEF DESCRIPTION OF THE DRAWINGS

FIG. 1 depicts a two-step, two-stage process for genetic modification in mammals. At the zygote stage, using Cas9-mediated homology-directed repair (HDR), a Bxb1 attP site is inserted in the CD68 locus, adjacent to the start codon (ATG) (A). At the two-cell stage, in the same embryo, using Bxb1 serine recombinase (integrase), Cas9-EGFP containing a Bxb1 attB site is integrated (B). The alleles containing the correct integration (B′) are used for humanization and human red blood cell (RBC) survival studies. EP, electroporation; RNP, ribonucleoprotein; ssODN, single-stranded donor oligonucleotides; MIJ, microinjection.

FIGS. 2A-2C show the effectiveness of expressing Cas9 in mice under the control of CD68 for genome editing in mice. Peritoneal macrophages were obtained from mice containing an expression cassette in the ROSA26 locus. The expression cassette contained nucleic acid sequences encoding human CD68 and Cas9, with both nucleic acid sequences being operably linked to the CD68 promoter, which is active in macrophages. FIG. 2A shows PCR amplicons of the Irf5 locus of macrophages transfected with scrambled sgRNAs (lanes 1 and 2) or two sgRNAs targeting sequences in Irf5 (lanes 3 and 4). Lanes 5 and 6 are non-template controls.

FIG. 2B shows the dropout (DO) region of the Irf5 gene that is deleted from the genome following targeted cleavage at both of the sites indicated by sgRNA 1 and sgRNA 2. FIG. 2C shows Sanger sequencing of the amplicon detected in lane 3 of FIG. 2A. sgRNA; single-stranded guide RNA; bp, base pairs; kb, kilobase; DO, dropout.

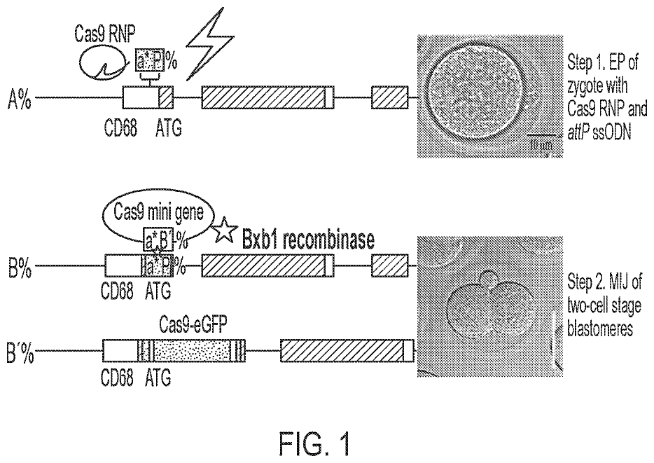

### DETAILED DESCRIPTION

Historically, the introduction of large transgenes in mice has been accomplished by either embryonic stem cell manipulation or more commonly by random transgenesis in the zygote, and more recently by programmable nuclease-mediated (e.g., CRISPR-mediated, e.g., Cas9-mediated) HDR directly in the zygote. Targeted transgenesis typically relies on the use of extensive homology arms flanking the donor transgene, resulting in even larger vector sizes. The use of such methods presents technical challenges in production and handling and requires extensive downstream work to mitigate any unintended consequences.

Further, programmable nuclease mediated HDR is very inefficient as the donor construct become greater than ˜4 kb. An alternative methodology, “random transgenesis” involves directly injecting DNA constructs into the zygote, on the hope that it will integrate into the genome and be functional. However, often animals (e.g., mammal, such as rodent, for example, mouse) models created using this approach suffer from positional effects, for example, disruption of native genes at the sites of integration(s), and aberrant transgene expression levels. Multicopy concatemers can lead to vast overexpression or with multiple insertions scattered over the genome, lead to segregation of the transgenes during breeding, with subsequent expression changes and instability of the required phenotype. Further, the often-coincident inclusion of elements from the prokaryotic plasmid backbone can result in transgene silencing, nullifying the utility of a potential mammalian model.

The present disclosure provides, in some aspects, a targeted transgenesis technology that results in a transgenic founder with a desired gene integrated into a chosen locus after a single generation, eliminating the need for an intervening generation to prepare for integration of the gene of interest.

### Single Generation Targeted Gene Integration

Some aspects of the present disclosure provide methods comprising: (a) delivering to a zygote a nucleic acid construct comprising a first attachment site (e.g., Bxb1 attP or attB); (b) culturing the zygote to produce a multi-cell embryo comprising the first integrase attachment site in the genome of the embryo; and (c) delivering to the embryo (i) a nucleic acid (e.g., DNA) comprising a second integrase attachment site and a sequence encoding a product (e.g., protein) of interest and (ii) a cognate integrase (e.g., Bxb1) or a nucleic acid (e.g., mRNA) encoding a cognate integrase to produce an engineered embryo comprising the sequence encoding the product of interest.

### One-Cell Stage Delivery

One step of the methods described herein includes delivering to a zygote (e.g., via electroporation) a nucleic acid comprising an integrase attachment site, which is integrated into the genome of embryo that develops from the zygote mediated by a programmable nuclease. Methods for facilitating genomic integration of nucleic acids are described elsewhere herein.

A zygote, also referred to as a fertilized oocyte, is a single-cell embryo, comprising a diploid cell formed by the fusion of two haploid gametes. In some embodiments, the zygote is a mammalian zygote. In some embodiments, the zygote is a non-human zygote (e.g., a commercial food animal, such as a cow, a sheep, a pig, a goat, a chicken, etc.). In some embodiments, the mammalian zygote is a rodent zygote. The rodent zygote may be, for example, a rat zygote or a mouse zygote. While primarily mouse zygotes are described herein, it should be understood that the methods of the present disclosure may be used to produce any transgenic animal.

In some embodiments, a mouse zygote is a NOD.Cg-Prkdc^{scid} Il2rg^{tm1Wjl}/SzJ (NSG®) mouse zygote. Other non-limiting examples of mouse strains that may be used include a NOD-Rag1^{null}, IL2rg^{null} (NRG), and NOD-Shi^{scid}Il2rgγ^{null} (NOG), C57BL/6J, C57BL/6NJ (5304), FVB/NJ (1800), BALB/cJ, BALB/cByJ, B6D2 (C57BL/6×DBA/2J), A/J (The Jackson Lab Stock No. 000646), 129S1/SvImJ (The Jackson Lab Stock No. 002448), NOD/ShiLtJ (The Jackson Lab Stock No. 001976), NZO/HiLtJ (The Jackson Lab Stock No. 002105), CAST/EiJ (The Jackson Lab Stock No. 000928), PWK/PhJ (The Jackson Lab Stock No. 003715), WSB/EiJ (The Jackson Lab Stock No. 001145), DBA2 (The Jackson Lab Stock No. 000671), and Collaborative Cross (CC) strains. Other strains are contemplated herein.

Following delivery of the integrase attachment site, the zygote may be cultured to produce a multi-cell embryo. Culturing a zygote under suitable conditions enables the zygote to undergo cell division to produce the multi-cell embryo. If nucleic acid (e.g., DNA) integration takes place prior to the first nuclear division, cells of the embryo will carry (in the genome) the integrase attachment site. If the integration occurs later, e.g., at the two-cell stage, the animals will be mosaic, carrying the attachment site in some of its cells. Conditions for culturing embryos, preimplantation, are known. See, e.g., Gardner D K & Lane M Mouse Molecular Embryology (2013) pp 167-182; and Tung E. W. Y., Winn L. M. (2019) Mouse Whole Embryo Culture. In: Hansen J., Winn L. (eds) Developmental Toxicology. Methods in Molecular Biology, vol 1965. Humana, New York, NY.

In some embodiments, the multi-cell embryo is a blastomere. In some embodiments, the multi-cell embryo comprises 2, 4, 8, or 16 cells, formed by repeated cell cleavage of the original zygote.

### Multi-Cell Stage Delivery

Another step of the methods described herein includes delivering to the multi-cell embryo (e.g., via microinjection), which now includes in its genome a first integrase attachment site, (i) a nucleic acid comprising a second integrase attachment site and a sequence encoding a product of interest and (ii) a cognate nuclease or a nucleic acid encoding a cognate integrase. Delivery of these nucleic acids and subsequent expression and activity of the encoded integrase results in integration of the sequence encoding a product of interest at the integrase attachment site that was delivered at the one-cell stage. This two-step/two-stage delivery process, performed on the same embryo, negates the need to establish a founder line prior to delivering a protein-coding sequences of interest, for example.

Following this step of the method, the embryo may be implanted into a pseudopregnant female mammal capable of giving birth to a progeny mammal. Pseudopregnancy describes a false pregnancy whereby all the signs and symptoms of pregnancy are exhibited, with the exception of the presence of a zygote. Mice become pseudopregnant following an estrus in which the female is bred by an infertile male, resulting in sterile mating.

### Integrase-Based Genetic Rearrangement

An integrase attachment site is delivered at the one-cell stage of the process provided herein. An integrase is an enzyme that catalyzes breaking and rejoining of nucleic acid (e.g., DNA) strands at specific points, referred to herein as attachment sites, thereby precisely rearranging the nucleic acids. Integrases belong to one of the two large families of site-specific recombinases, referred to as serine recombinases and tyrosine recombinases according to the nucleophilic active site amino acid residue that attacks specific DNA phosphodiesters to cleave strands.

In some embodiments, the integrase is Bxb1, which is a serine recombinase encoded by the Bxb1 mycobacteriophage. This serine recombinase may be used for the introduction of any human, mouse (or any other species), or synthetic construct to a mammalian genome. In nature, the Bxb1 integrase functions to perform DNA strand exchange between unique attachment sites in the phage (“attP”) and its bacterial host (“attB”) during its lysogenic phase. Each attachment site is shorter than 50 nucleotide base pairs (bp) in length, which is ideal for use in molecular cloning as well as for insertion into host genomes using, for example, gene editing techniques. The Bxb1 integrase works in eukaryotic cells and does not require any additional host factors to function. Further, it has been shown to function at high efficiency in cells, is unidirectional and has no detectable pseudo sites in the mouse genome. The Bxb1 system also lends itself to enhancement, as the two central dinucleotides in the attachment sites are solely responsible for the specificity of the recombination event. These combined attributes render this system useful for directly modifying mammalian (e.g., mouse) zygotes.

While the present disclosure focuses primarily on Bxb1 integrase, it should be understood that other site-specific recombinases and attachment sites may be used. In some embodiments, a different serine recombinase is used, for example, gamma-delta resolvase, Tn3 resolvase, φC31 integrase, or R4 integrase. In some embodiments, a tyrosine recombinase is used, for example, Cre recombinase or FLP recombinase. A further embodiment utilizes custom built, or synthetic integrases designed to target novel sites.

Although the Bxb1 integrase attachment DNA sites are relatively small (<50 bp), the reaction is highly selective for these sites and is also strongly directional (see, e.g., Singh A et al. PLoS Genetics 2013; 9(5): e1003490). The Bxb1 attB sites show at least seven unique and specific optimal variations, plus a further nine suboptimal variations in an internal dinucleotide recognition sequence, allowing the same Bxb1 recombinase enzyme to use a series of different constructs at the same time each with its specific dinucleotide address (see. e.g., Ghosh P et al. J. Mol Biol. 2006; 349:331-348). Thus, contemplated herein is the use of Bxb1 attP sites and modified attP* sites (e.g., modified relative to the sequence of SEQ ID NO: 1), as well as the use of Bxb1 attB sites and modified attB* sites (e.g., modified relative to the sequence of SEQ ID NO: 3)

It should be understood, unless noted otherwise, that the Bxb1 attachment site that is introduced to the host animal genome (“genomic attachment site”) may be a Bxb1 attP site, a modified Bxb1 attP site, a Bxb1 attB site, modified Bxb1 attB site, or any combination thereof. The corresponding donor polynucleotide to be inserted into the Bxb1 attachment site should include another Bxb1 attachment site(s). Thus, if the Bxb1 attachment site in the genome is a Bxb1 attP site, the corresponding polynucleotide (e.g., circular donor DNA) to be inserted into the genomic Bxb1 attachment site should include a Bxb1 attB site; and if the Bxb1 attachment site in the genome is a Bxb1 attB site, the corresponding polynucleotide to be inserted into the genomic Bxb1 attachment site should include a Bxb1 attP site.

In some embodiments, a single Bxb1 attachment site is introduced in a genomic locus of an embryo. For example, the Bxb1 attachment site may be selected from attP attachment sites, modified attP* attachment sites, attB attachment sites, and modified attB* attachment sites.

In other embodiments, two (at least two) Bxb1 attachment sites are introduced in a genomic locus of the embryo, which may be referred to herein as a first Bxb1 attachment site and a second Bxb1 attachment site. The first and second Bxb1 attachment sites, in some embodiments, are selected from attP attachment sites, modified attP* attachment sites, attB attachment sites, and modified attB* attachment sites. The first and second Bxb1 attachment sites may be adjacent to each other (with no intervening nucleotide sequence) or they may be separated from each other by a certain number of nucleotides. The number of nucleotides separating the two Bxb1 attachment sites may vary, provided, in some embodiments, that each Bxb1 attachment site is within the same target gene locus (e.g., within the CD68 locus). Thus, in some embodiments, any two (e.g., a first and second) Bxb1 attachments sites are separated from each other by at least 1, at least 2, at least 5, at least 10, at least 25, at least 50, at least 100, at least 150, at least 200, at least 250, at least 300, at least 350, at least 400, at least 450, at least 500, at least 1000, at least 1500, or at least 2000 nucleotide base pairs (bp). In some embodiments, any two (e.g., a first and second) Bxb1 attachments sites are separated from each other by 1 to 500 bp, 1 to 1000 bp, 1 to 1500 bp, 1 to 2000 bp, 1 to 2500 bp, or 1 to 3000 nucleotide base pairs (bp). For example, any two Bxb1 attachments sites may be separated from each other by 1 to 450 bp, 1 to 400 bp, 1 to 350 bp, 1 to 300 bp, 1 to 250 bp, 1 to 200 bp, 1 to 150 bp, 1 to 100 bp, 1 to 50 bp, 5 to 450 bp, 5 to 400 bp, 5 to 350 bp, 5 to 300 bp, 5 to 250 bp, 5 to 200 bp, 5 to 150 bp, 5 to 100 bp, 5 to 50 bp, 10 to 450 bp, 10 to 400 bp, 10 to 350 bp, 10 to 300 bp, 10 to 250 bp, 10 to 200 bp, 10 to 150 bp, 10 to 100 bp, 10 to 50 bp, 50 to 450 bp, 50 to 400 bp, 50 to 350 bp, 50 to 300 bp, 50 to 250 bp, 50 to 200 bp, 50 to 150 bp, 50 to 100 bp, 100 to 450 bp, 100 to 400 bp, 100 to 350 bp, 100 to 300 bp, 100 to 250 bp, 100 to 200 bp, or 100 to 150 bp.

In some embodiments, the Bxb1 attachment site(s) is/are located in or near the start codon (ATG) of an endogenous gene. For example, the normal transcriptional regulatory elements of an endogenous gene may be “intercepted” by including a Bxb1 attachment site near the start codon of the gene, then integrating the gene of interest (via Bxb1 integrase) such that transcription of the gene of interest is under the control of the transcriptional regulatory elements of the endogenous gene. In this way, in some embodiments, the integrase attachment site is operably linked to an endogenous promoter of a gene of interest. In some embodiments, the integrase attachment site is upstream from (5′) and in frame with a transcriptional start codon.

In some embodiments it the integrase attachment site is downstream from and in frame with a transcriptional start codon with the objective of producing a hybrid protein, for example, to increase stability.

This gene interception permits both spatial and temporal control of gene expression, depending on the location and timing of activity of endogenous promoter. Advantageously, any cell type can be targeted. Non-limiting examples of cell types include stem cells, red blood cells, white blood cells, platelets, macrophages, neutrophils, nerve cells, muscle cells, cartilage cells, bone cells, skin cells, endothelial cells, epithelial cells, and fat cells.

Exogenous promoters may also be used to drive expression of a product (e.g., protein of interest).

The Bxb1 attachment site(s), in some embodiments, is/are located in a safe harbor locus, which is an open chromatin region of a genome. Genomic safe harbors (GSHs) are sites in the genome able to accommodate the integration of new genetic material in a manner that ensures that the newly inserted genetic elements: (i) function predictably and (ii) do not cause alterations of the host genome posing a risk to the host cell or organism (see, e.g., Papapetrou E P and Schambach A Mol Ther 2016; 24(4): 678-684).

Non-limiting examples of safe harbor loci that may be used as provided herein include the AAVS1 locus, ROSA26 locus, the Hip11 locus, the Hprt locus, and the Tigre locus. Other safe harbor loci may be used as provided herein.

In some embodiments, the integrase attachment site is flanked by nucleotide sequences homologous to nucleotide sequences in the genome of the zygote. In some embodiments, the nucleotide sequences are homologous to a gene of interest in the genome of the zygote. In some embodiments, the gene of interest is a gene that is gene that is expressed in a particular cell type. In some embodiments, the gene of interest is CD68.

One homology arm is located to the left (5′) of the integrase site(s) (the left homology arm) and another homology arm is located to the right (3′) of the integrase site(s) (the right homology arm). Homology arms are regions of the ssDNA that are homologous to regions of genomic DNA located in the genomic (e.g., CD68) locus. These homology arms enable homologous recombination between the ssDNA donor and the genomic locus, resulting in insertion of the Bxb1 attachment site(s) into the genomic locus, as discussed below (e.g., via CRISPR/Cas9-mediated homology directed repair (HDR)).

The homology arms may vary in length. For example, each homology arm (the left arm and the right homology arm) may have a length of 20 nucleotide bases to 1000 nucleotide bases. In some embodiments, each homology arm has a length of 20 to 200, 20 to 300, 20 to 400, 20 to 500, 20 to 600, 20 to 700, 20 to 800, or 20 to 900 nucleotide bases. In some embodiments, each homology arm has a length of 20, 30, 40, 50, 60, 70, 80, 90, 100, 150, 200, 250, 300, 350, 400, 450, 500, 550, 600, 650, 700, 750, 800, 850, 900, 950, or 1000 nucleotide bases. In some embodiments, the length of one homology arm differs from the length of the other homology arm. For example, one homology arm may have a length of 20 nucleotide bases, and the other homology arm may have a length of 50 nucleotide bases. In some embodiments, the donor DNA is single stranded. In some embodiments, the donor DNA is double stranded. In some embodiments, the donor DNA is modified, e.g., via phosphorothioation. Other modifications may be made.

The concentration of nucleic acid comprising a Bxb1 attachment site (e.g., landing pad) may vary. In some embodiments, the concentration is 500 ng/μl to 5000 ng/μl, or 500 ng/μl to 3000 ng/μl. For example, the concentration may be 500, 600, 700, 800, 900, 1000, 1100, 1200, 1300, 1400, 1500, 1600, 1700, 1800, 1900, 2000, 2100, 2200, 2300, 2400, or 3500 500 ng/μl to 5000 ng/μl.

The concentration of nucleic acid (e.g., mRNA) encoding a Bxb1 integrase (or another integrase) may vary. In some embodiments, the concentration is 50 ng/μl to 500 ng/μl, or 50 ng/μl to 200 ng/μl. For example, the concentration may be 50, 60, 70, 80, 90, 100, 110, 120, 130, 140, 150, 160, 170, 180, 190, or 200 ng/μl.

In some embodiments, a concentration of 2 to 100 ng/ul of Bxb1 protein is delivered with or without mRNA encoding Bxb1 (e.g., simultaneously or consecutively).

### Engineered Nucleic Acids, Delivery Methods, and Integration Methods

The nucleic acids provided herein, in some embodiments, are engineered. An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a nucleic acid is a complementary DNA (cDNA). cDNA is synthesized from a single-stranded RNA (e.g., messenger RNA (mRNA) or microRNA (miRNA)) template in a reaction catalyzed by reverse transcriptase.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences).

A promoter is a nucleotide sequence to which RNA polymerase binds to initial transcription (e.g., ATG). Promoters are typically located directly upstream from (at the 5′ end of) a transcription initiation site. In some embodiments, a promoter is an endogenous promoter. An endogenous promoter is a promoter that naturally occurs in that host animal.

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

An exon is a region of a gene that codes for amino acids. An intron (and other non-coding DNA) is a region of a gene that does not code for amino acids.

A nucleotide sequence encoding a product of interest, in some embodiments, has a length of 200 base pairs (bp) to 100 kilobases (kb). The nucleotide sequence, in some embodiments, has a length of at least 10 kb. For example, the nucleotide sequence may have a length of at least 15 kb, at least 20 kb, at least 25 kb, at least 30 kb, or at least 35 kb. In some embodiments, the nucleotide sequence has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb.

Any one of the nucleic acids provided herein may have a length of 200 bp to 500 kb, 200 bp to 250 kb, or 200 bp to 100 kb. A nucleic acid, in some embodiments, has a length of at least 10 kb. For example, a nucleic acid may have a length of at least 15 kb, at least 20 kb, at least 25 kb, at least 30 kb, at least 35 kb, at least 50 kb, at least 100 kb, at least 200 kb, at least 300 kb, at least 400 kb, or at least 500 kb. In some embodiments, the donor polynucleotide has a length of 10 to 500 kb, 20 to 400 kb, 10 to 300 kb, 10 to 200 kb, or 10 to 100 kb. In some embodiments, a nucleic acid has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb. A nucleic acid polynucleotide may be circular or linear.

The concentration of nucleic acid (e.g., DNA minicircle) encoding a product of interest may vary. In some embodiments, the concentration is 10 ng/μl to 1000 ng/μl or 10 ng/μl to 100 ng/μl. For example, the concentration may be 10, 20, 30, 40, 50, 60, 70, 80, 90, or 100 ng/μl.

Vectors used for delivery of a nucleic acid include minicircles, plasmids, bacterial artificial chromosomes (BACs), and yeast artificial chromosomes. It should be understood, however, that a vector may not be needed. For example, a circularized or linearized nucleic acid may be delivered to an embryo without its vector backbone. Vector backbones are small (˜4 kb), while donor DNA to be circularized can range from >100 bp to 50 kb, for example.

In some embodiments, a DNA minicircle is a plasmid derivative that has been freed from all prokaryotic vector parts (e.g., no longer contains a bacterial plasmid backbone comprising antibiotic resistance markers and/or bacterial origins of replication). Methods of producing DNA minicircles are well-known in the art. For example, a parental plasmid that comprises a bacterial backbone and the eukaryotic inserts, including the transgene to be expressed, may be produced in a specialized E. coli strain that expresses a site-specific recombinase protein. Recombination sites flank the eukaryotic inserts in the parental plasmid, so that when the activity of the recombinase protein (non-Bxb1) is induced by methods such as, but not limited to, arabinose induction, glucose induction, etc., the bacterial backbone is excised from the eukaryotic insert, resulting in a eukaryotic DNA minicircle and a bacterial plasmid.

Methods for delivering nucleic acids to rodent embryos for the production of transgenic rodents include, but are not limited to, electroporation (see, e.g., Wang W et al. J Genet Genomics 2016; 43(5):319-27; WO 2016/054032; and WO 2017/124086, each of which is incorporated herein by reference), DNA microinjection (see, e.g., Gordon and Ruddle, Science 1981: 214: 1244-124, incorporated herein by reference), embryonic stem cell-mediated gene transfer (see, e.g., Gossler et al., Proc. Natl. Acad. Sci. 1986; 83: 9065-9069, incorporated herein by reference), and retrovirus-mediated gene transfer (see, e.g., Jaenisch, Proc. Natl. Acad. Sci. 1976; 73: 1260-1264, incorporated herein by reference), any of which may be used as provided herein.

Engineered nucleic acids, such as guide RNAs, donor polynucleotides, and other nucleic acid coding sequences, for example, may be introduced to a genome of an embryo using any suitable method. The present application contemplates the use of a variety of gene editing technologies, for example, to introduce nucleic acids into the genome of an embryo to produce a transgenic rodent. Non-limiting examples include programmable nuclease-based systems, such as clustered regularly interspaced short palindromic repeat (CRISPR) systems, zinc-finger nucleases (ZFNs), and transcription activator-like effector nucleases (TALENs). See, e.g., Carroll D Genetics. 2011; 188(4): 773-782; Joung J K et al. Nat Rev Mol Cell Biol. 2013; 14(1): 49-55; and Gaj T et al. Trends Biotechnol. 2013 July; 31(7): 397-405, each of which is incorporated by reference herein.

In some embodiments, a CRISPR system is used to edit the genome of rodent (e.g., mouse) embryos provided herein. See, e.g., Harms D W et al., Curr Protoc Hum Genet. 2014; 83: 15.7.1-15.7.27; and Inui M et al., Sci Rep. 2014; 4: 5396, each of which are incorporated by reference herein). For example, Cas9 mRNA or protein and one or multiple guide RNAs (gRNAs) can be delivered, e.g., injected or electroporated, directly into rodent embryos at the one-cell (zygote) stage to facilitate homology directed repair (HDR) to introduce an engineered nucleic acid into the genome.

The CRISPR/Cas system is a naturally occurring defense mechanism in prokaryotes that has been repurposed as a RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target (e.g., gene) to be modified. Thus, one can change the genomic target of the Cas protein by simply changing the target sequence present in the gRNA. In some embodiments, the Cas9 endonuclease is from Streptococcus pyogenes (NGG PAM) or Staphylococcus aureus (NNGRRT or NNGRR(N) PAM), although other Cas9 homologs, orthologs, and/or variants (e.g., evolved versions of Cas9) may be used, as provided herein. Additional non-limiting examples of RNA-guided nucleases that may be used as provided herein include Cpf1 (TTN PAM); SpCas9 D1135E variant (NGG (reduced NAG binding) PAM); SpCas9 VRER variant (NGCG PAM); SpCas9 EQR variant (NGAG PAM); SpCas9 VQR variant (NGAN or NGNG PAM); Neisseria meningitidis (NM) Cas9 (NNNNGATT PAM); Streptococcus thermophilus (ST) Cas9 (NNAGAAW PAM); and Treponema denticola (TD) Cas9 (NAAAAC). In some embodiments, the CRISPR-associated endonuclease is selected from Cas9, Cpf1, C2c1, and C2c3. In some embodiments, the Cas nuclease is Cas9.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence. See, e.g., Jinek et al., Science, 2012; 337: 816-821 and Deltcheva et al., Nature, 2100; 471: 602-607, each of which is incorporated by reference herein.

In some embodiments, the RNA-guided nuclease and the gRNA are complexed to form a ribonucleoprotein (RNP), prior to delivery to an embryo.

The concentration of RNA-guided nuclease or nucleic acid encoding the RNA-guided nuclease may vary. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl. In some embodiments, the concentration is 100 ng/μl to 500 ng/μl, or 200 ng/μl to 500 ng/μl.

The concentration of gRNA may also vary. In some embodiments, the concentration is 200 ng/μl to 2000 ng/μl. For example, the concentration may be 200, 300, 400, 500, 600, 700, 800, 900, 1000, 1100, 1200, 1300, 1400, 1500, 1600, 1700, 1700, 1900, or 2000 ng/μl. In some embodiments, the concentration is 500 ng/μl to 1000 ng/μl. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl.

In some embodiments, the ratio of concentration of RNA-guided nuclease or nucleic acid encoding the RNA-guided nuclease to the concentration of gRNA is 2:1. In other embodiments, the ratio of concentration of RNA-guided nuclease or nucleic acid encoding the RNA-guided nuclease to the concentration of gRNA is 1:1.

In some embodiments, the product of interest is a programmable nuclease. A programmable nuclease, such as any of the CRISPR/Cas nucleases, zinc finger nucleases, or transcription activator-like effector nucleases (TALENs) described herein, when expressed from the genome of a cell, may be used to edit another site in the genome. In some embodiments, the product of interest is an RNA-guided nuclease. Exemplary RNA-guided nucleases include Cas9, Cpf1, C2c1, and C2c3. In some embodiments, the product of interest is Cas9. In some embodiments, the method further comprises delivering to the embryo a guide RNA (gRNA) targeting a nucleotide sequence of interest. In some embodiments, the nucleotide sequence of interest is an interferon regulatory factor 5 (Irf5) gene. In some embodiments, the product of interest is a zinc finger nuclease (ZFN). In some embodiments, the product of interest is a transcription activator-like effector nuclease (TALEN).

### EXAMPLES

### Example 1

This Example describes a two-step, two-stage genomic modification strategy to generate a mouse strain expressing a recombinant protein from an endogenous promoter in a single generation. The CD68 gene locus in NSG® (NOD.Cg-Prkdc^{scid} Il2rg^{tm1Wjl}/SzJ) mice was used as an example. The locus was ‘intercepted’ with a sequence encoding Cas9-EGFP to provide expression in a tissue-specific manner, directed by the mouse CD68 promoter in a living transgenic mouse. This was done by sequential addition: first, a nucleic acid comprising a Bxb1 attP attachment site (also referred to as a landing pad) was delivered to a mouse zygote (one-cell stage) by electroporation; and second, a nucleic acid encoding Bxb1 integrase and a nucleic acid comprising a Bxb1 attB site and a sequence encoding Cas9-EGFP was delivered to the same embryo by microinjection at the two-cell stage.

On day one (1), fertilized oocytes from an NSG® mouse strain were electroporated (EP) to insert a landing pad (Bxb1-attP_GA) (SEQ ID NO: 5) into the CD68 locus on chromosome 11 using a Cas9-guide complex, to facilitate oligo-mediated homology-directed repair (HDR). To form the Cas9-guide complex, Streptococcus pyogenes Cas9 (SpCas9) protein (417 ng/μl) was incubated with a CD68-targeting guide RNA (gRNA) (SEQ ID NO: 6) (864 ng/μl) and an ˜150 base pair (bp) donor oligonucleotide with an ˜50 bp attP site flanked by ˜ 50 bp homology arms (2097 ng/μl) for 15 minutes at 37° C., then placed on ice (4° C.). The embryos were then cultured overnight using standard methods known in the art (e.g., cultured overnight in microdrops of COOK RCVL under oil (Parrafin); in incubators at 37 C, 5/5/90 (5% CO₂/5% O₂/90% N₂; any appropriate culture medium (e.g., KSOM+AA) can be substituted for COOK RCVL).

On day two (2), Bxb1 mRNA (100 ng/μl), donor DNA prepared as a minicircle (30 ng/μl, and RNAsin (0.2 U/μl) were combined in TE (10 mM Tris/0.1 mM EDTA/pH7.5) and microinjected into the nucleus of both cells. The donor DNA was prepared as a 6,061 bp minicircle containing the corresponding Bxb1-attB_GA site, 3× FLAG®-tagged Cas9-EGFP coding sequence (separated by a 2A peptide), followed by the woodchuck hepatitis post-transcriptional regulatory element (WPRE) and a bovine growth hormone polyadenylation signal.

The microinjected embryos were then transferred to pseudopregnant females and carried to term. At wean, tail tissue biopsies were taken from the offspring and genotyped by PCR and Sanger sequencing. Sequencing results showed that 7 out of 15 (47%) mice had attP sites correctly inserted into the CD68 locus and 2 out of 15 (13%) mice had the desired Cas9-EGFP construct correctly integrated at the CD68 locus. Positive founder candidates were then bred to establish the new genetically engineered mouse strain.

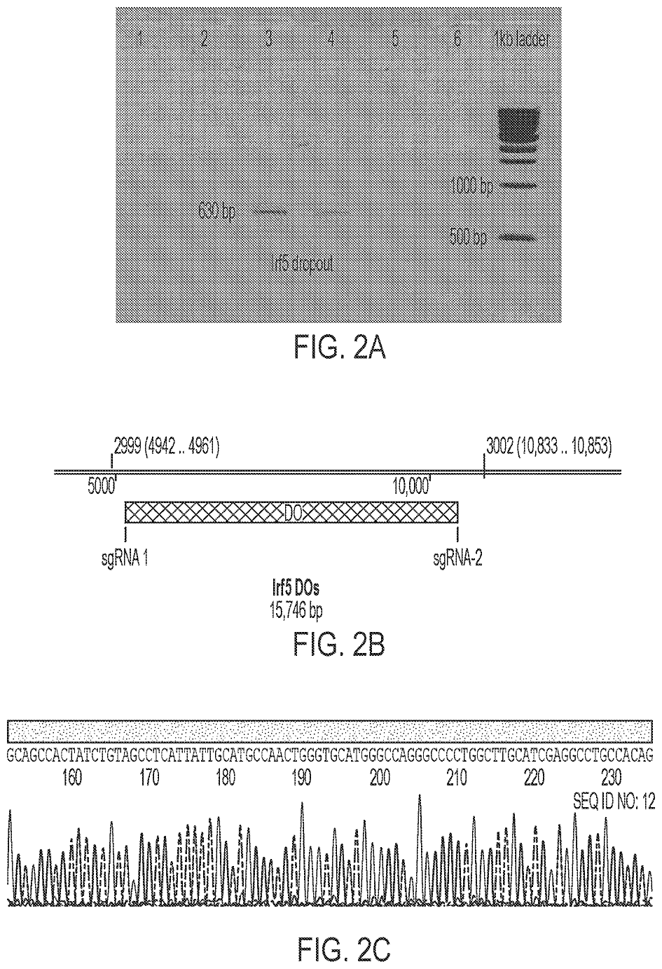

Next, an expression cassette comprising a mouse CD68 promoter operably linked to both (i) a nucleic acid sequence encoding human CD68; and (ii) a nucleic acid sequence encoding Cas9, was inserted into the ROSA26 locus of mouse chromosome 6. Peritoneal macrophages were isolated, then transfected with one of two sgRNAs targeting the mouse Irf5 locus, or two scrambled sgRNAs containing a random rearrangement of the nucleotide sequences of the Irf5 locus-targeting sgRNAs. The first Irf5 locus-targeting sgRNA contained the sequence CGAGGCAUGGUCCCAGCC (SEQ ID NO: 7), and the second Irf5 locus-targeting sgRNA contained the sequence UUGCAGCCCGGUUGCUGC (SEQ ID NO: 8). Transfection with either of these sgRNAs, but not with either of the scrambled sgRNAs, resulted in the generation of a gene dropout event in the Irf5 locus (FIG. 2A). Following cleavage of the Irf5 locus at a sequence targeted by either of these sgRNAs, non-homologous end joining (NHEJ) resulted in a gene dropout event due to insertion or deletion of one or more bases during DNA repair. The location of this gene dropout event is shown in FIG. 2B. Sanger sequencing of the Irf5 locus confirmed this dropout event, as evidenced by the heterogeneity of an amplicon containing the targeted region, indicating the presence of multiple different insertions and deletions in the targeted sequence (FIG. 2C). These results indicate that expression of Cas9 from a nucleic acid sequence inserted into a safe harbor locus, such as the ROSA26 locus on mouse chromosome 6, allows for targeted cleavage, and thus genome editing by homology-directed repair (HDR), in mice.

### Example 2

In this Example, the mouse host cell receptor angiotensin-converting enzyme 2 (mAce2) gene (e.g., SEQ ID NO: 9) is ‘intercepted’ with an open reading frame encoding human ACE2 (huACE2) (e.g., SEQ ID NOs: 10 or 11). This is done by sequential addition: first, a nucleic acid comprising a Bxb1 attP attachment site is delivered to a mouse zygote (one-cell stage) by electroporation; and second, a nucleic acid encoding Bxb1 integrase and a nucleic acid comprising a Bxb1 attB site and a sequence encoding huACE2 is delivered to the same embryo by microinjection at the two-cell stage, as described in Example 1.

The microinjected embryos are then transferred to pseudopregnant females and carried to term. At wean, tail tissue biopsies are taken from the offspring and genotyped by PCR and Sanger sequencing. Positive founder candidates are then bred to establish the new genetically engineered mouse strain.

### Example 3

In this Example, the mouse albumin gene is ‘intercepted’ with an open reading frame encoding human albumin. This is done by sequential addition: first, a nucleic acid comprising a Bxb1 attP attachment site is delivered to a mouse zygote (one-cell stage) by electroporation; and second, a nucleic acid encoding Bxb1 integrase and a nucleic acid comprising a Bxb1 attB site and a sequence encoding human albumin is delivered to the same embryo by microinjection at the two-cell stage, as described in Example 1.

The microinjected embryos are then transferred to pseudopregnant females and carried to term. At wean, tail tissue biopsies are taken from the offspring and genotyped by PCR and Sanger sequencing. Positive founder candidates are then bred to establish the new genetically engineered mouse strain.

SEQUENCES
Bxb1 attP site
(SEQ ID NO: 1)
GGTTTGTCTGGTCAACCACCGCGGTCTCAGTGGTGTACGGTACAAACC
Bxb1 attP* site
(SEQ ID NO: 2)
GGTTTGTCTGGTCAACCACCGCGGACTCAGTGGTGTACGGTACAAACC
Bxb1 attB site
(SEQ ID NO: 3)
GGCTTGTCGACGACGGCGGTCTCCGTCGTCAGGATCAT
Bxb1 attB* site
(SEQ ID NO: 4)
GGCTTGTCGACGACGGCGGACTCCGTCGTCAGGATCAT.
Bxb1 Donor Oligonucleotide (Example 1)
(SEQ ID NO: 5)
G*A*TTGAGGAAGGAACTGGTGTAGCCTAGCTGGTCTGAGCATCTCTGCCATGCGGTTTGTCTG
GTCAACCACCGCGGACTCAGTGGTGTACGGTACAAACCGGCTCCCTGTGTGTCTGATCTTGCTA
GGACCGCTTATAGGTAAGGAGA*A*A
*phosphorothioate modifications
CD68 gRNA target
(SEQ ID NO: 6)
GACACACAGGGAGCCGCATGG
Irf5 gRNA 1
(SEQ ID NO: 7)
CGAGGCAUGGUCCCAGCC
Irf5 gRNA 2
(SEQ ID NO: 8)
UUGCAGCCCGGUUGCUGC
Mouse Ace2 Exon 2 - site of human ACEs insertion is underlined
(SEQ ID NO: 9)
TGCCCAACCCAAGTTCAAAGGCTGATGAGAGAGAAAAACTCATGAAGAGATTTTACTCTAGGGA
AAGTTGCTCAGTGGATGGGATCTTGGCGCACGGGGAAAGATGTCCAGCTCCTCCTGGCTCCTTC
TCAGCCTTGTTGCTGTTACTACTGCTCAGTCCCTCACCGAGGAAAATGCCAAGACATTTTTAAA
CAACTTTAATCAGGAAGCTGAAGACCTGTCTTATCAAAGTTCACTTGCTTCTTGGAATTATAAT
ACTAACATTACTGAAGAAAATGCCCAAAAGATG
HuACE2 CDS + FLAG ® TAG CDS (2442 bp)
(SEQ ID NO: 10)
ATGGCAAGCTCTTCCTGGCTCCTTCTCAGCCTTGTTGCTGTAACTGCTGCTCAGTCCACCATTG
AGGAACAGGCCAAGACATTTTTGGACAAGTTTAACCACGAAGCCGAAGACCTGTTCTATCAAAG
TTCACTTGCTTCTTGGAATTATAACACCAATATTACTGAAGAGAATGTCCAAAACATGAATAAT
GCTGGGGACAAATGGTCTGCCTTTTTAAAGGAACAGTCCACACTTGCCCAAATGTATCCACTAC
AAGAAATTCAGAATCTCACAGTCAAGCTTCAGCTGCAGGCTCTTCAGCAAAATGGGTCTTCAGT
GCTCTCAGAAGACAAGAGCAAACGGTTGAACACAATTCTAAATACAATGAGCACCATCTACAGT
ACTGGAAAAGTTTGTAACCCAGATAATCCACAAGAATGCTTATTACTTGAACCAGGTTTGAATG
AAATAATGGCAAACAGTTTAGACTACAATGAGAGGCTCTGGGCTTGGGAAAGCTGGAGATCTGA
GGTCGGCAAGCAGCTGAGGCCATTATATGAAGAGTATGTGGTCTTGAAAAATGAGATGGCAAGA
GCAAATCATTATGAGGACTATGGGGATTATTGGAGAGGAGACTATGAAGTAAATGGGGTAGATG
GCTATGACTACAGCCGCGGCCAGTTGATTGAAGATGTGGAACATACCTTTGAAGAGATTAAACC
ATTATATGAACATCTTCATGCCTATGTGAGGGCAAAGTTGATGAATGCCTATCCTTCCTATATC
AGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTTGGTCTT
CCTAATATGACTCAAGGATTCTGGGAAAATTCCATGCTAACGGACCCAGGAAATGTTCAGAAAG
CAGTCTGCCATCCCACAGCTTGGGACCTGGGGAAGGGCGACTTCAGGATCCTTATGTGCACAAA
GGTGACAATGGACGACTTCCTGACAGCTCATCATGAGATGGGGCATATCCAGTATGATATGGCA
TATGCTGCACAACCTTTTCTGCTAAGAAATGGAGCTAATGAAGGATTCCATGAAGCTGTTGGGG
AAATCATGTCACTTTCTGCAGCCACACCTAAGCATTTAAAATCCATTGGTCTTCTGTCACCCGA
TTTTCAAGAAGACAATGAAACAGAAATAAACTTCCTGCTCAAACAAGCACTCACGATTGTTGGG
ACTCTGCCATTTACTTACATGTTAGAGAAGTGGAGGTGGATGGTCTTTAAAGGGGAAATTCCCA
AAGACCAGTGGATGAAAAAGTGGTGGGAGATGAAGCGAGAGATAGTTGGGGTGGTGGAACCTGT
GCCCCATGATGAAACATACTGTGACCCCGCATCTCTGTTCCATGTTTCTAATGATTACTCATTC
ATTCGATATTACACAAGGACCCTTTACCAATTCCAGTTTCAAGAAGCACTTTGTCAAGCAGCTA
AACATGAAGGCCCTCTGCACAAATGTGACATCTCAAACTCTACAGAAGCTGGACAGAAACTGTT
CAATATGCTGAGGCTTGGAAAATCAGAACCCTGGACCCTAGCATTGGAAAATGTTGTAGGAGCA
AAGAACATGAATGTAAGGCCACTGCTCAACTACTTTGAGCCCTTATTTACCTGGCTGAAAGACC
AGAACAAGAATTCTTTTGTGGGATGGAGTACCGACTGGAGTCCATATGCAGACCAAAGCATCAA
AGTGAGGATAAGCCTAAAATCAGCTCTTGGAGATAAAGCATATGAATGGAACGACAATGAAATG
TACCTGTTCCGATCATCTGTTGCATATGCTATGAGGCAGTACTTTTTAAAAGTAAAAAATCAGA
TGATTCTTTTTGGGGAGGAGGATGTGCGAGTGGCTAATTTGAAACCAAGAATCTCCTTTAATTT
CTTTGTCACTGCACCTAAAAATGTGTCTGATATCATTCCTAGAACTGAAGTTGAAAAGGCCATC
AGGATGTCCCGGAGCCGTATCAATGATGCTTTCCGTCTGAATGACAACAGCCTAGAGTTTCTGG
GGATACAGCCAACACTTGGACCTCCTAACCAGCCCCCTGTTTCCATATGGCTGATTGTTTTTGG
AGTIGTGATGGGAGTGATAGTGGTTGGCATTGTCATCCTGATCTTCACTGGGATCAGAGATCGG
AAGAAGAAAAATAAAGCAAGAAGTGGAGAAAATCCTTATGCCTCCATCGATATTAGCAAAGGAG
AAAATAATCCAGGATTCCAAAACACTGATGATGTTCAGACCTCCTTTGATTACAAGGATGACGA
CGATAAGTGA
HuACE2 + FLAG TAG (813 AA, 93.4 kDa predicted)
(SEQ ID NO: 11)
MASSSWLLLSLVAVTAAQSTIEEQAKTFLDKFNHEAEDLFYQSSLASWNYNTNITEENVQNMNN
AGDKWSAFLKEQSTLAQMYPLQEIQNLTVKLQLQALQQNGSSVLSEDKSKRLNTILNTMSTIYS
TGKVCNPDNPQECLLLEPGLNEIMANSLDYNERLWAWESWRSEVGKQLRPLYEEYVVLKNEMAR
ANHYEDYGDYWRGDYEVNGVDGYDYSRGQLIEDVEHTFEEIKPLYEHLHAYVRAKLMNAYPSYI
SPIGCLPAHLLGDMWGRFWTNLYSLTVPFGQKPNIDVTDAMVDQAWDAQRIFKEAEKFFVSVGL
PNMTQGFWENSMLTDPGNVQKAVCHPTAWDLGKGDFRILMCTKVTMDDFLTAHHEMGHIQYDMA
YAAQPFLLRNGANEGFHEAVGEIMSLSAATPKHLKSIGLLSPDFQEDNETEINFLLKQALTIVG
TLPFTYMLEKWRWMVFKGEIPKDQWMKKWWEMKREIVGVVEPVPHDETYCDPASLFHVSNDYSF
IRYYTRTLYQFQFQEALCQAAKHEGPLHKCDISNSTEAGQKLFNMLRLGKSEPWTLALENVVGA
KNMNVRPLLNYFEPLFTWLKDQNKNSFVGWSTDWSPYADQSIKVRISLKSALGDKAYEWNDNEM
YLFRSSVAYAMRQYFLKVKNQMILFGEEDVRVANLKPRISFNFFVTAPKNVSDIIPRTEVEKAI
RMSRSRINDAFRLNDNSLEFLGIQPTLGPPNQPPVSIWLIVFGVVMGVIVVGIVILIFTGIRDR
KKKNKARSGENPYASIDISKGENNPGFQNTDDVQTSFDYKDDDDK

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A method comprising:
(a) delivering to a zygote a nucleic acid comprising a first integrase attachment site;
(b) culturing the zygote to produce a multi-cell embryo comprising the first integrase attachment site in the genome of the embryo; and
(c) delivering to the embryo a nucleic acid comprising a second integrase attachment site and a sequence encoding a product of interest to produce an engineered embryo.

2. The method of claim 1 further comprising delivering to the embryo a cognate integrase or a nucleic acid encoding a cognate integrase.

3. The method of claim 1, wherein the zygote comprises a sequence encoding a cognate integrase, wherein the cognate integrase is integrated into the genome of the zygote.

4. The method of any one of the preceding claims further comprising implanting the engineered embryo into a pseudopregnant female mammal capable of giving birth to a progeny mammal.

5. The method of any one of the preceding claims, wherein the delivering to the zygote is via electroporation.

6. The method of any one of the preceding claims, wherein the delivering to the embryo is via microinjection into each cell of the embryo.

7. The method of any one of the preceding claims, wherein the multi-cell embryo is a two-cell embryo.

8. The method of any one of the preceding claims, wherein the cognate integrase is Bxb1.

9. The method of claim 8, wherein the first integrase attachment site of (a) is a Bxb1 attP attachment site, and the second integrase attachment site of (c) is a Bxb1 attB attachment site.

10. The method of claim 8, wherein the first integrase attachment site of (a) is a Bxb1 attB attachment site, and the second integrase attachment site of (c) is a Bxb1 attP attachment site.

11. The method of claim 10, wherein the first integrase attachment site of (b) is operably linked to an endogenous promoter of a gene of interest.

12. The method of claim 11, wherein the first integrase attachment site of (b) is upstream from (5′) and in frame with a transcriptional start codon.

13. The method of claim 11, wherein the first integrase attachment site of (b) is downstream from (5′) and in frame with a transcriptional start codon.

14. The method of any one of the preceding claims, wherein the first integrase attachment site of (a) is flanked by nucleotide sequences homologous to nucleotide sequences in the genome of the zygote.

15. The method of any one of the preceding claims, wherein the nucleic acid of (c) is a DNA minicircle.

16. The method of any one of the preceding claims, wherein the nucleic acid encoding the cognate integrase is a messenger RNA (mRNA).

17. The method of any one of the preceding claims wherein (a) further comprises delivering to the zygote a programmable nuclease, wherein the nucleic acid in (a) is used as a template for modifying the genome of the zygote following cleavage of the genome by the programmable nuclease, thereby introducing the first integrase attachment site into the genome of the zygote.

18. The method of claim 17, wherein the programmable nuclease is an RNA-guided nuclease and the method further comprises delivering to the zygote (i) an RNA-guided nuclease or a nucleic acid encoding the RNA-guided nuclease and (ii) a guide RNA (gRNA) targeting a nucleotide sequence of interest.

19. The method of claim 18, wherein the RNA-guided nuclease and the gRNA form a ribonucleoprotein.

20. The method of claim 18 or 19, wherein the RNA-guided nuclease is Cas9.

21. The method of claim 17, wherein the programmable nuclease is a zinc finger nuclease (ZFN).

22. The method of claim 17, wherein the programmable nuclease is a transcription activator-like effector nuclease (TALEN).

23. The method of any one of the preceding claims, wherein the zygote is a mammalian zygote.

24. The method of claim 23, wherein the mammalian zygote is a rodent zygote.

25. The method of claim 24, wherein the rodent zygote is a rat zygote.

26. The method of claim 24, wherein the rodent zygote is a mouse zygote.

27. The method of claim 26, wherein the mouse zygote is a NOD.Cg-Prkdc^{scid} Il2rg^{tm1Wjl}/SzJ (NSG®) mouse zygote.

28. The method of any one of the preceding claims, wherein the gene of interest is CD68.

29. The method of any one of the preceding claims further comprising breeding progeny mammals birthed by the pseudopregnant female mammal.

30. The method of any one of claims 24-29, wherein the rodent zygote is a mouse zygote and the endogenous promoter is a mouse host cell receptor angiotensin-converting enzyme 2 (mAce2) promoter.

31. The method of any one of the preceding claims, wherein the product of interest is human host cell receptor angiotensin-converting enzyme 2 (huACE2).

32. A progeny mammal produced by the method of claim 29, wherein the progeny mammal is a rodent, optionally a mouse.

33. A rodent comprising the engineered embryo produced by the method of any one of the preceding claims.

34. A method comprising administering a candidate prophylactic or therapeutic agent to the progeny mammal of claim 32.

35. The method of claim 34, wherein the candidate agent is convalescent human serum, a human vaccine, or an antimicrobial agent, optionally an antibacterial agent and/or an antiviral agent.

36. The method of claim 34 or 35 further comprising infecting the mouse with SARS-CoV-2.

37. The method of claim 36 further comprising assessing efficacy of the agent for preventing SARS-CoV-2 infection and/or development of COVID-19.

38. The method of any one of the preceding claims, wherein the product of interest is a programmable nuclease.

39. The method of claim 38, wherein the product of interest is an RNA-guided nuclease.

40. The method of claim 39, wherein the product of interest is Cas9.

41. The method of claim 39 or 40, wherein the method further comprises delivering to the embryo a guide RNA (gRNA) targeting a nucleotide sequence of interest.

42. The method of claim 41, wherein the nucleotide sequence of interest is an interferon regulatory factor 5 (Irf5) gene.

43. The method of claim 38, wherein the product of interest is a zinc finger nuclease (ZFN).

44. The method of claim 38, wherein the product of interest is a transcription activator-like effector nuclease (TALEN).

45. The method of any one of the preceding claims, wherein the first integrase attachment site is located in a safe harbor locus of the genome.

46. The method of claim 45, wherein the safe harbor locus is a ROSA26 locus, an AAVS1 locus, a Hip11 locus, an Hprt locus, or a Tigre locus.
